(19) United States
(12) Patent Application Publication
(10) Pub. No.: US 2024/0268776 A1
(43) Pub. Date: Aug. 15, 2024

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Tomoko TAKI, Kanagawa (JP)

(73) Assignee: FUJIFILM Corporation, Tokyo (JP)

(21) Appl. No.: 18/645,539

(22) Filed: Apr. 25, 2024

Related U.S. Application Data
(63) Continuation of application No. 17/071,536, filed on Oct. 15, 2020.

(30) Foreign Application Priority Data
Oct. 18, 2019 (JP) 2019-191405

(51) Int. Cl. A61B 6/50; A61B 6/00; A61B 6/42; G01T 1/161

(52) U.S. Cl. CPC A61B 6/505; A61B 6/4241; A61B 6/482; G01T 1/161

(57) ABSTRACT

An information processing apparatus includes a soft part image generation unit that generates a soft part image representing a soft part region with a soft tissue of a subject from a first radiographic image and a second radiographic image acquired by radiation having different energy distributions transmitted through the subject, and a muscle mass derivation unit that derives a muscle mass based on a pixel value for each pixel of the soft part region of the soft part image.

### CROSS-REFERENCE TO RELATED APPLICATION

This application is a Continuation of U.S. patent application Ser. No. 17/071,536 filed Oct. 15, 2020, which claims benefit of priority to Japanese Patent Application No. 2019-191405 filed Oct. 18, 2019, the entire contents of which are incorporated herein by reference.

### BACKGROUND

### Field of the Invention

A technique of the present disclosure relates to an information processing apparatus, an information processing method, and an information processing program.

### Related Art

A Dual X-ray Absorptiometry (DXA) method is known as a representative bone mineral quantitation method use to diagnose bone density in a bone disease, such as osteoporosis. The DXA method is a method that calculates a bone mineral content from pixel values of a radiographic image obtained by imaging with radiation having two kinds of energy levels using the fact that radiation, which is incident on a human body and is transmitted through the human body, is subjected to attenuation characterized by a mass attenuation coefficient μ (cm²/g) depending on a substance (for example, bone) forming the human body, density ρ (g/cm³) of the substance, and a thickness t (cm) of the substance.

Furthermore, a radiography apparatus that comprises two radiation detectors including a plurality of pixels, which accumulate electric charge according to irradiated radiation and the two radiation detectors are disposed to be stacked is known. In addition, a technique that measures a bone mineral content of a subject using each electric signal according to a dose of radiation irradiated to each radiation detector in this kind of radiography apparatus is known (see JP2018-015453A).

Furthermore, a technique that derives a ratio of each of a fat tissue and a lean tissue of each pixel in a DXA image is known (see JP2019-063499A). In the technique described in JP2019-063499A, a fat tissue ratio and a lean tissue (muscle, non-fat, and non-mineral tissues) ratio are measured by analyzing a combination of low-energy and high-energy DXA images.

Incidentally, there is known an injury or ailment for which a correspondence relationship between a risk of a disease, a disease level, or the like and a muscle mass is recognized. It is desirable to ascertain a muscle mass of a subject from a viewpoint of prevention, medical examination for such an injury or ailment. In particular, it is desirable to ascertain a local muscle mass in a part (tissue) of the subject related to the injury or ailment (disease).

### SUMMARY

The present disclosure has been accomplished in consideration of the above circumstances, and an object of the present disclosure is to provide an information processing apparatus, an information processing method, and an information processing program capable of ascertaining a local muscle mass of a subject.

In order to achieve the above-described object, a first aspect of the present disclosure provides an information processing apparatus comprising a soft part image generation unit that generates a soft part image representing a soft part region with a soft tissue of a subject from a first radiographic image and a second radiographic image acquired by radiation having different energy distributions transmitted through the subject, and a muscle mass derivation unit that derives a muscle mass based on a pixel value for each pixel of the soft part region of the soft part image.

According to a second aspect of the present disclosure, in the information processing apparatus according to the first aspect of the present disclosure, the muscle mass derivation unit derives a muscle mass with a tissue other than a fat tissue in the soft tissue as a muscle tissue.

According to a third aspect of the present disclosure, the information processing apparatus according to the first aspect or the second aspect of the present disclosure further comprises a bone part image generation unit that generates a bone part image representing a bone part region with a bone tissue of the subject from the first radiographic image and the second radiographic image, and a bone mineral content derivation unit that derives a bone mineral content based on a pixel value for each pixel of the bone part region of the bone part image.

According to a fourth aspect of the present disclosure, in the information processing apparatus according to any one aspect of the first aspect to the third aspect of the present disclosure, the muscle mass derivation unit derives the muscle mass of a predetermined part of the subject. The information processing apparatus further comprises a specification unit that, based on correspondence relationship information representing a correspondence relationship between disease information representing an affection risk of a predetermined disease or a disease level of the predetermined disease and a muscle mass of the predetermined part and the muscle mass derived by the muscle mass derivation unit, specifies the affection risk or the disease level.

According to a fifth aspect of the present disclosure, in the information processing apparatus according to the third aspect of the present disclosure, the muscle mass derivation unit derives the muscle mass of a predetermined part of the subject. The information processing apparatus further comprises a specification unit that, based on correspondence relationship information representing a correspondence relationship between disease information representing an affection risk of a predetermined disease or a disease level of the predetermined disease and a muscle mass of the predetermined part, the muscle mass derived by the muscle mass derivation unit, and the bone mineral content derived by the bone mineral content derivation unit, specifies the disease information.

According to a sixth aspect of the present disclosure, in the information processing apparatus according to the fourth aspect or the fifth aspect of the present disclosure, the predetermined part is a part of a lower limb, the predetermined disease is diabetes, and the disease information is the affection risk of the diabetes.

According to a seventh aspect of the present disclosure, in the information processing apparatus according to the fourth aspect or the fifth aspect of the present disclosure, the predetermined part is a part of a limb or a part of a whole body, the predetermined disease is sarcopenia, and the disease information is the disease level of the sarcopenia.

According to an eighth aspect of the present disclosure, in the information processing apparatus according to any one aspect of the first aspect to the third aspect of the present disclosure, the muscle mass derivation unit derives the muscle mass of a predetermined part of the subject. The information processing apparatus further comprises a specification unit that, based on correspondence relationship information representing a correspondence relationship between the muscle mass of the predetermined part or muscle information regarding muscle strength according to the muscle mass of the predetermined part and a fall rate of the subject and the muscle mass derived by the muscle mass derivation unit, specifies the fall rate.

According to a ninth aspect of the present disclosure, in the information processing apparatus according to the third aspect of the present disclosure, the muscle mass derivation unit derives the muscle mass of a predetermined part of the subject. The information processing apparatus further comprises a specification unit that, based on correspondence relationship information representing a correspondence relationship between the muscle mass of the predetermined part or muscle information regarding muscle strength according to the muscle mass of the predetermined part and a fall rate of the subject, the muscle mass derived by the muscle mass derivation unit, and the bone mineral content derived by the bone mineral content derivation unit, specifies the fall rate.

According to a tenth aspect of the present disclosure, in the information processing apparatus according to the eighth aspect or the ninth aspect of the present disclosure, the predetermined part is at least one of a lower limb or a pelvis.

According to an eleventh aspect of the present disclosure, the information processing apparatus according to any one aspect of the first aspect to the tenth aspect of the present disclosure further comprises a muscle mass distribution image generation unit that generates a muscle mass distribution image representing a distribution of the muscle mass based on the muscle mass.

According to a twelfth aspect of the present disclosure, in the information processing apparatus according to any one aspect of the first aspect to the eleventh aspect of the present disclosure, the first radiographic image is acquired by a first radiation detector between the first radiation detector and a second radiation detector superimposed in an irradiation direction of radiation transmitted through the subject, and the second radiographic image is acquired by the second radiation detector.

According to a thirteenth aspect of the present disclosure, in the information processing apparatus according to any one aspect of the first aspect to the eleventh aspect of the present disclosure, the first radiographic image is acquired by a radiation detector that is irradiated with radiation having a first energy distribution transmitted through the subject, and the second radiographic image is acquired by the radiation detector that is irradiated with radiation having a second energy distribution different from the first energy distribution transmitted through the subject.

Furthermore, in order to achieve the above-described object, a fourteenth aspect of the present disclosure provides an information processing method comprising generating a soft part image representing a soft part region with a soft tissue of a subject from a first radiographic image and a second radiographic image acquired by radiation having different energy distributions transmitted through the subject, and deriving a muscle mass based on a pixel value for each pixel of the soft part region of the soft part image.

In addition, in order to achieve the above-described object, a fifteenth aspect of the present disclosure provides an information processing program that causes a computer to execute generating a soft part image representing a soft part region with a soft tissue of a subject from a first radiographic image and a second radiographic image acquired by radiation having different energy distributions transmitted through the subject, and deriving a muscle mass based on a pixel value for each pixel of the soft part region of the soft part image.

According to the present disclosure, it is possible to ascertain a local muscle mass of a subject.

### BRIEF DESCRIPTION OF THE DRAWINGS

FIG. 1 is a schematic block diagram showing an example of the configuration of a radiography system of a first embodiment.

FIG. 2 is a diagram showing an example of the hardware configuration of an information processing apparatus of the first embodiment.

FIG. 3 is a functional block diagram showing an example of the functional configuration of the information processing apparatus of the first embodiment.

FIG. 4 is a diagram showing an example of energy spectrums of radiation after transmission through a muscle tissue and radiation after transmission through a fat tissue.

FIG. 5 is a flowchart showing an example of a flow of information processing that is executed by the information processing apparatus of the first embodiment.

FIG. 6 is a functional block diagram showing an example of the functional configuration of an information processing apparatus of a second embodiment.

FIG. 7 is a diagram showing a relationship between a body thickness of a subject and contrast of a bone tissue and a soft tissue.

FIG. 8 is a diagram showing an example of a look-up table that is used in the second embodiment.

FIG. 9 shows an example of correspondence relationship information representing a correlative relationship between a muscle mass and a fall rate in the second embodiment.

FIG. 10 is a flowchart showing an example of a flow of information processing that is executed by the information processing apparatus of the second embodiment.

FIG. 11 is a functional block diagram showing an example of the functional configuration of an information processing apparatus of a third embodiment.

FIG. 12 is a diagram showing an example of a muscle mass distribution image that is generated by a muscle mass distribution image generation unit.

FIG. 13 is a flowchart showing an example of a flow of information processing that is executed by the information processing apparatus of the third embodiment.

### DETAILED DESCRIPTION OF

Hereinafter, examples of an embodiment for implementing the technique of the present disclosure will be described in detail referring to the drawings. In the drawings, the substantially same or equivalent components or portions are represented by the same reference numerals.

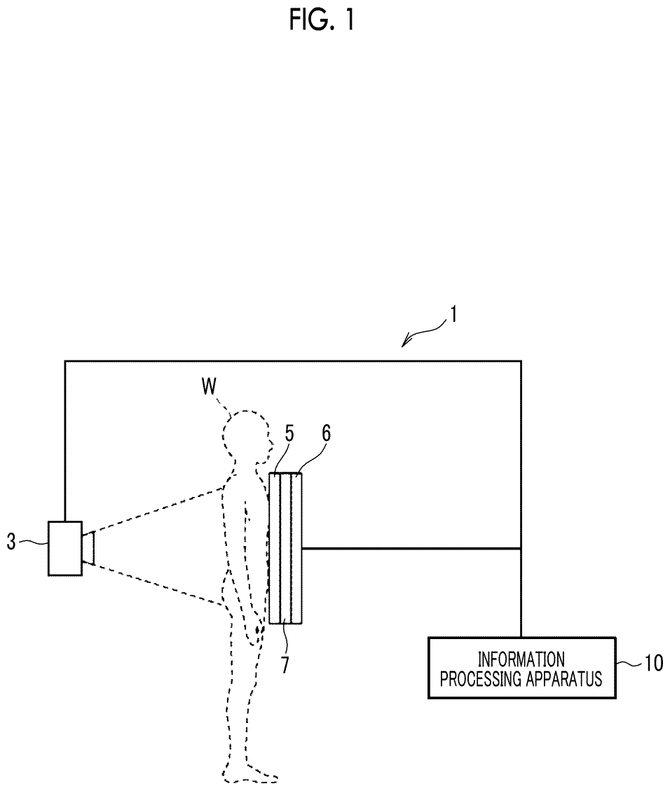

FIG. 1 is a schematic block diagram showing an example of the configuration of a radiography system 1 of the embodiment. The radiography system 1 comprises a radiation source 3, a first radiation detector 5, a second radiation detector 6, a radiation-energy conversion filter 7, and an information processing apparatus 10.

Each of the first radiation detector 5 and the second radiation detector 6 generates a radiographic image based on radiation emitted from the radiation source 3 and transmitted through a subject W. Each of the first radiation detector 5 and the second radiation detector 6 may have a form of a so-called flat panel detector (FPD) in which a radiographic image signal is read by turning on and off a thin film transistor (TFT) switch. In this case, each of the first radiation detector 5 and the second radiation detector 6 may be a direct radiation detector that is directly irradiated with radiation to generate an electric charge or an indirect radiation detector that converts radiation into visible light once and converts the visible light into an electric charge signal. Each of the first radiation detector 5 and the second radiation detector 6 may be a radiation detector to which a Computed Radiography (CR) technique for reading an image recorded on an imaging plate through irradiation of a laser beam is applied. The radiation-energy conversion filter 7 is configured of a metal plate, such as a copper plate, capable of absorbing a specific energy component included in radiation.

A radiographic image is captured in a state in which the first radiation detector 5, the radiation-energy conversion filter 7, and the second radiation detector 6 are superimposed in this order from a side close to the radiation source 3 (subject W), whereby one-shot energy subtraction is realized. That is, with irradiation with radiation from the radiation source 3 once, two radiographic images having different energy distributions are acquired from the first radiation detector 5 and the second radiation detector 6.

In the first radiation detector 5, a first radiographic image G1 of the subject W according to low-energy radiation including so-called soft rays is acquired. Furthermore, in the second radiation detector 6, a second radiographic image G2 of the subject W according to high-energy radiation without soft rays is acquired. Each of the first radiographic image G1 and the second radiographic image G2 is input to the information processing apparatus 10.

In the radiography system 1 of the embodiment, in a case where the subject W is imaged, a scattered ray elimination grid that eliminates scattered ray components of radiation transmitted through the subject W is not used. For this reason, primary ray components and scattered ray components of radiation transmitted through the subject W are included in each of the first radiographic image G1 and the second radiographic image G2.

The information processing apparatus 10 has a function of deriving a local muscle mass of the subject W based on the first radiographic image G1 and the second radiographic image G2 acquired on the subject W.

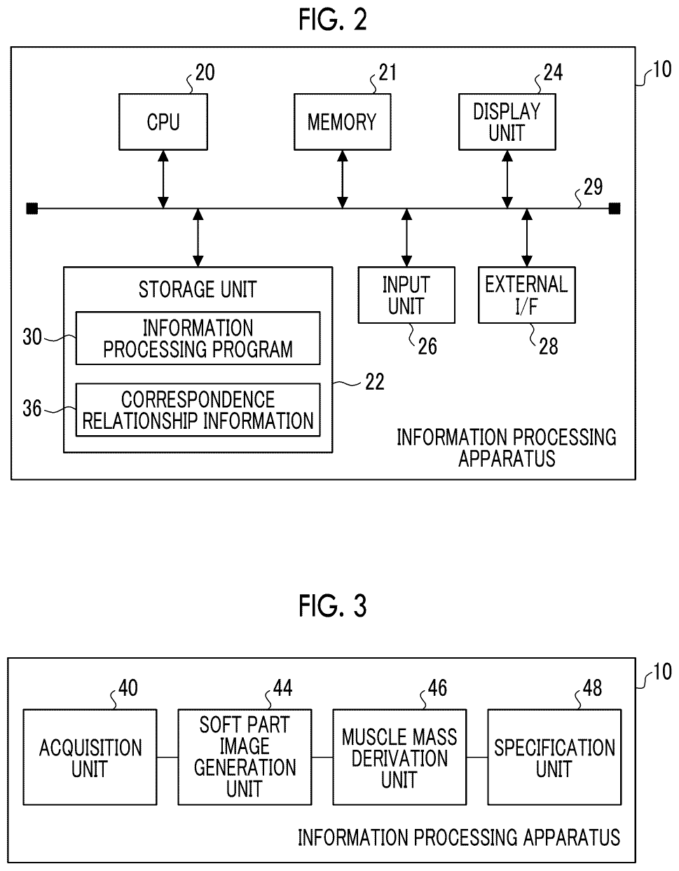

FIG. 2 is a diagram showing an example of the hardware configuration of the information processing apparatus 10 of the embodiment. The information processing apparatus 10 comprises a central processing unit (CPU) 20, a memory 21, a storage unit 22, a display unit 24, such as a liquid crystal display, an input unit 26, such as a keyboard and a mouse, and an external interface (I/F) 28. The CPU 20, the memory 21, the storage unit 22, the display unit 24, the input unit 26, and the external I/F 28 are connected to a bus 29. The first radiation detector 5 and the second radiation detector 6 are connected to the external I/F 28. The information processing apparatus 10 may configure, for example, a personal computer or a server computer.

The storage unit 22 is realized by a hard disk drive (HDD), a solid state drive (SSD), a flash memory, or the like. An information processing program 30 is stored in the storage unit 22 as a storage medium. The CPU 20 reads the information processing program 30 from the storage unit 22, then, develops the information processing program 30 to the memory 21, and executes the developed information processing program 30. Furthermore, a correspondence relationship information 36 described below is stored in the storage unit 22.

FIG. 3 is a functional block diagram showing an example of the functional configuration of the information processing apparatus 10. The information processing apparatus 10 comprises an acquisition unit 40, a soft part image generation unit 44, a muscle mass derivation unit 46, and a specification unit 48. The information processing apparatus 10 functions as the acquisition unit 40, the soft part image generation unit 44, the muscle mass derivation unit 46, and the specification unit 48 by the CPU 20 executing the information processing program 30.

The acquisition unit 40 acquires the first radiographic image G1 and the second radiographic image G2 acquired by radiation having different energy distributions transmitted through the subject W. Specifically, the acquisition unit 40 acquires image data representing the first radiographic image G1 output from the first radiation detector 5, and acquires image data representing the second radiographic image G2 output from the second radiation detector 6. In imaging of the first radiographic image G1 and the second radiographic image G2, imaging conditions, such as an irradiation dose, a tube voltage, and a source-to-image receptor distance (SID) of radiation from the radiation source 3, are set. The set imaging conditions are stored in the storage unit 22 in association with each of the first radiographic image G1 and the second radiographic image G2. The SID used herein represents a distance between the radiation source 3 and a detection surface of radiation in the first radiation detector 5 in a case of the first radiation detector 5, and represents a distance between the radiation source 3 and a detection surface of radiation in the second radiation detector 6 in a case of the second radiation detector 6.

The soft part image generation unit 44 generates a soft part image Gs representing a soft part region with a soft tissue of the subject W from the first radiographic image G1 and the second radiographic image G2 acquired by the acquisition unit 40. In the embodiment, the “soft tissue” of the subject W refers to a tissue other than a bone tissue, and specifically includes a muscle tissue, a fat tissue, blood, and moisture.

As an example, the soft part image generation unit 44 of the embodiment performs weighting subtraction between corresponding pixels for the first radiographic image G1 and the second radiographic image G2 as shown in Expression (1) described below, thereby generating the soft part image Gs in which only the soft tissues of the subject W included in the first radiographic image G1 and the second radiographic image G2 are extracted. In Expression (1) described below, μs is a weighting factor, and x and y are coordinates of each pixel of the soft part image Gs.

$$Gs ( x , y ) = G 1 ( x , y ) - μ s \times G 2 ( x , y ) \qquad ( 1 )$$

As described above, scattered ray components other than primary ray components of radiation transmitted through the subject W are included in each of the first radiographic image G1 and the second radiographic image G2. For this reason, it is preferable that the scattered ray components are eliminated from the first radiographic image G1, the second radiographic image G2, and the soft part image Gs. Information for eliminating the scattered ray components from the first radiographic image G1, the second radiographic image G2, and the soft part image Gs is not particularly limited. For example, the soft part image generation unit 44 may apply the method described in JP2015-043959A to eliminate the scattered ray components from the first radiographic image G1, the second radiographic image G2, and the soft part image Gs.

For example, a method in which a plurality of pieces of data for scattered ray correction obtained in advance by calibration using a phantom simulating a human body and a flat plate-shaped radiation shielding member with a pinhole, through which radiation is transmitted, in a center portion is stored in the storage unit 22, and the soft part image generation unit 44 eliminates scattered rays from the first radiographic image G1, the second radiographic image G2, and the soft part image Gs using the data for scattered ray correction may be applied. The amount of scattered rays to be generated is different depending on the above-described imaging conditions or conditions, such as a body thickness of the subject W and a composition (a ratio of muscle and fat) of the soft tissue of the subject. For this reason, it is preferable that the data for scattered ray correction is acquired in advance according to each condition using a plurality of kinds of phantoms according to various conditions.

A distance (SID) between the radiation source 3 and each of the first radiation detector 5 and the second radiation detector 6, or the like is different. For this reason, a spread of scattered rays, called a point spread function (PSF), or intensity of scattered rays is different and a content of scattered rays is different between the first radiographic image G1 and the second radiographic image G2. For this reason, the data for scattered ray correction may be stored in association with each of the first radiographic image G1 and the second radiographic image G2 or processing of correcting a difference in scattered ray content between the first radiographic image G1 and the second radiographic image G2 may be executed.

As described above, since the amount of scattered ray components included in each of the first radiographic image G1 and the second radiographic image G2 is different according to the body thickness of the subject W, it is preferable that the soft part image generation unit 44 eliminates scattered rays in consideration of the body thickness of the subject W.

A method in which the soft part image generation unit 44 derives the body thickness of the subject W is not particularly limited, and a body thickness distribution T(x,y) of the subject W may be derived, for example, using a method described in JP2015-043959A. In the following description, an example of a method of deriving the body thickness distribution T(x,y) of the subject W will be described. In the example, although a form using the first radiographic image G1 acquired by the radiation detector 5 on the side close to the subject W will be described, the present disclosure is not limited to this form, and the second radiographic image G2 may be used.

First, the soft part image generation unit 44 acquires a virtual model K of the subject W having an initial body thickness distribution T0(x,y). The virtual model K is data that virtually represents the subject W and has the body thickness compliant with the initial body thickness distribution T0(x,y) in association with a coordinate position of each pixel of the first radiographic image G1. The virtual model K of the subject W having the initial body thickness distribution T0(x,y) may be stored in advance in the storage unit 22.

Next, the soft part image generation unit 44 generates an image, in which an estimated primary ray image of a primary ray image to be obtained by imaging the virtual model K and an estimated scattered ray image of a scattered ray image to be obtained by imaging the virtual model K are composed, as an estimated image of the first radiographic image G1 obtained by imaging the subject W based on the virtual model K.

Next, the soft part image generation unit 44 corrects the initial body thickness distribution T0(x,y) of the virtual model K such that a difference between the estimated image and the first radiographic image G1 becomes small. The soft part image generation unit 44 repeats the generation of the estimated image and the correction of the body thickness distribution until the difference between the estimated image and the first radiographic image G1 satisfies a predetermined end condition. The soft part image generation unit 44 derives, as the body thickness distribution T(x,y) of the subject W, the body thickness distribution in a case where the end condition is satisfied.

The muscle mass derivation unit 46 derives a muscle mass based on a pixel value for each pixel of a soft part region in the soft part image Gs generated by the soft part image generation unit 44. As described above, the soft tissue includes a muscle tissue, a fat tissue, blood, and moisture. As an example, the muscle mass derivation unit 46 of the embodiment regards a tissue other than a fat tissue in the soft tissue as a muscle tissue. That is, the muscle mass derivation unit 46 of the embodiment handles a non-fat tissue including blood and moisture in addition to a muscle tissue as a muscle tissue.

The muscle mass derivation unit 46 separates muscle and fat from the soft part image Gs using a difference in energy characteristic between the muscle tissue and the fat tissue.

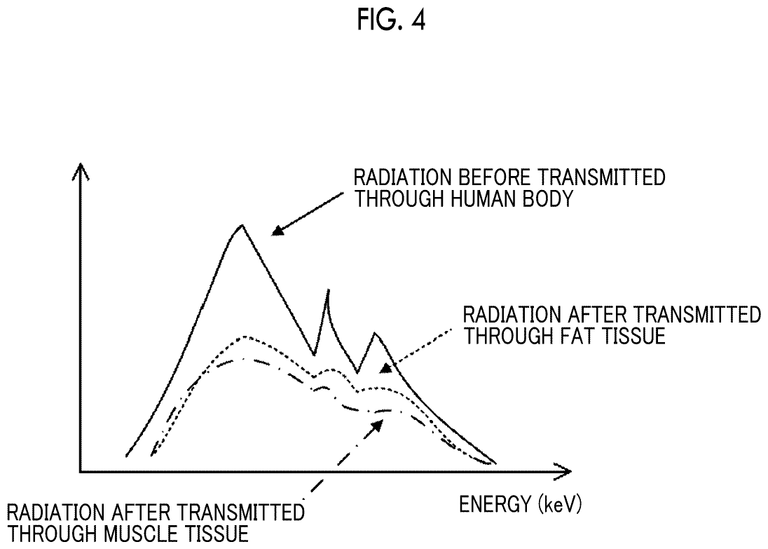

As shown in FIG. 4, radiation after transmission through the subject W is lowered compared to radiation before incidence on the subject W, which is a human body. Furthermore, since the muscle tissue and the fat tissue absorb different energy and have different attenuation coefficients, an energy spectrum is different between radiation after transmission through the muscle tissue and radiation after transmission through the fat tissue out of radiation after transmission through the subject W. As shown in FIG. 4, the energy spectrum of radiation that is transmitted through the subject W and irradiates each of the first radiation detector 5 and the second radiation detector 6 depends on a body composition of the subject, specifically, the ratio between the muscle tissue and the fat tissue. Since the fat tissue more easily transmits radiation than the muscle tissue, in a case where the ratio of the muscle tissue is greater than the fat tissue, the dose of radiation after transmission through the human body becomes small.

Accordingly, the muscle mass derivation unit 46 of the embodiment separates muscle and fat from the soft part image Gs using the difference in energy characteristic between the muscle tissue and the fat tissue described above. That is, the muscle mass derivation unit 46 generates a muscle image and a fat image from the soft part image Gs. Furthermore, the muscle mass derivation unit 46 derives a muscle mass of each pixel based on pixel values of the muscle image.

Although a specific method in which the muscle mass derivation unit 46 separates muscle and fat from the soft part image Gs is not limited, as an example, the muscle mass derivation unit 46 of the embodiment generates the muscle image from the soft part image Gs according to Expressions (2) and (3) described below. Specifically, first, the muscle mass derivation unit 46 derives a muscle rate rm(x,y) at each pixel position (x,y) in the soft part image Gs according to Expression (2) described below. In Expression (2) described below, μm is a weighting factor according to the attenuation coefficient of the muscle tissue, and μf is a weighting factor according to the attenuation coefficient of the fat tissue. Furthermore, Δ(x,y) represents a density difference distribution. The density difference distribution is a distribution of a difference in density on an image evident from a density obtained in a case where radiation reaches the first radiation detector 5 or the second radiation detector 6 without being transmitted through the subject W. The distribution of difference in density on the image is calculated by subtracting a density of each pixel in a region of the subject W from a density of a directly irradiated region in the soft part image Gs obtained in a case where the first radiation detector 5 or the second radiation detector 6 are directly irradiated with radiation.

$$r m ( x , y ) = \frac{μ f - \frac{Δ ( x , y )}{T ( x , y )}}{μ f - μ m} \qquad ( 2 )$$

In addition, the muscle mass derivation unit 46 generates the muscle image from the soft part image Gs according to Expression (3) described below. In Expression (3) described below, x and y are coordinates of each pixel of a muscle image Gm.

$$Gm ( x , y ) = r m ( x , y ) \times G s ( x , y ) \qquad ( 3 )$$

The muscle mass derivation unit 46 may derive an absolute amount of muscle as a muscle mass or may derive a ratio of muscle in each pixel.

The specification unit 48 specifies an affection risk of a predetermined disease or a disease level of the predetermined disease based on a muscle mass of the predetermined part derived by the muscle mass derivation unit 46 and the correspondence relationship information 36 and outputs information representing a specification result to the display unit 24. In the embodiment, the muscle mass of the predetermined part refers to an average value of muscle masses of respective pixels of a region in the soft part image Gs corresponding to the predetermined part. For example, the predetermined part is a thigh, an average value of muscle masses of respective pixels of a region in the soft part image Gs corresponding to the thigh is handled as a muscle mass of the thigh.

The correspondence relationship information 36 is information representing a correspondence relationship between disease information representing the affection risk of the predetermined disease or the disease level of the predetermined disease and the muscle mass of the predetermined part.

In general, there is a disease known that a muscle mass and an affection risk have a relationship. For example, it is known that muscle takes a part of glucose in blood and adjustment of a blood glucose level is performed. For this reason, in a case where the mass of muscle decreases, the blood glucose level increases, and an affection risk of diabetes increases. In particular, there is a tendency to correspond to a decrease in muscle mass of a lower limb. For this reason, in regard to diabetes, information representing a correspondence relationship between the muscle mass of the thigh, which is a part of the lower limb as a predetermined part, and the affection risk of diabetes is set as correspondence relationship information 36. As a specific example, for each age, an average value of the muscle masses of the thigh is set as a reference value, and correspondence relationship information 36 in which a higher affection risk is associated as the muscle mass is smaller than the reference value and has a greater divergence amount from the reference value is exemplified.

Furthermore, for example, in a case of sarcopenia, it is known that, in a case where illness is progressing, that is, in a case where the disease level becomes high, the muscle mass, in particular, a muscle mass of a limb tends to decrease. For this reason, in regard to sarcopenia, information representing a correspondence relationship between the muscle mass of the limb as a predetermined part and the disease level (progression degree) of sarcopenia is set as correspondence relationship information 36. As a specific example, for each age, an average value of the muscle masses of the limb is set as a reference value, and correspondence relationship information 36 in which a higher disease level is associated as the muscle mass is smaller than the reference value and has a greater divergence amount from the reference value is exemplified.

The information processing apparatus 10 may store the correspondence relationship information 36 according to a plurality of kinds of diseases in the storage unit 22 or may store the correspondence relationship information 36 for a specific disease in the storage unit 22.

In this way, the specification unit 48 of the embodiment specifies the affection risk of the predetermined disease or the disease level of the predetermined disease using the correspondence relationship information 36 according to the part (imaging part) of the subject captured in the first radiographic image G1 and the second radiographic image G2. A method in which the specification unit 48 specifies the part of the subject captured in the first radiographic image G1 and the second radiographic image G2 is not particularly limited. In a case where an imaging part is associated with the first radiographic image G1 and the second radiographic image G2 along with imaging conditions, a part of the subject may be specified based on the associated imaging part. Alternatively, for example, a part of the subject may be specified from information relating to a bone, such as a shape or a size of a bone captured in the first radiographic image G1 and the second radiographic image G2. Furthermore, for example, a form may be made in which the specification unit 48 acquires a part of the subject input by a user, such as a physician, through the input unit 26.

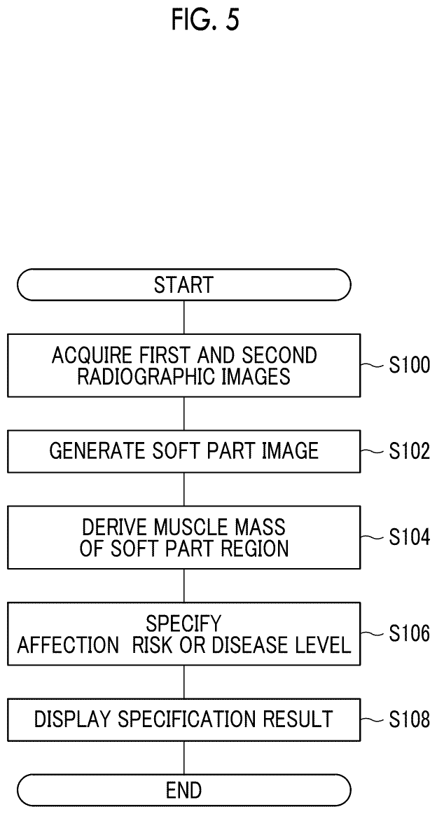

Next, the operation of the information processing apparatus 10 of the embodiment will be described. FIG. 5 is a flowchart showing an example of a flow of information processing that is executed by the CPU 20 executing the information processing program 30. The information processing program 30 is executed, for example, in a case where an instruction to start execution is input by the user through the input unit 26.

In Step S100 shown in FIG. 5, as described above, the acquisition unit 40 acquires the first radiographic image G1 and the second radiographic image G2 obtained by imaging the subject W from the first radiation detector 5 and the second radiation detector 6 of the radiography system 1, respectively.

In next Step S102, as described above, the soft part image generation unit 44 performs weighting subtraction shown in Expression (1) described above between the corresponding pixels in the first radiographic image G1 and the second radiographic image G2, thereby generating the soft part image Gs representing the soft part region with the soft tissue of the subject.

In next Step S104, as described above, the muscle mass derivation unit 46 performs weighting subtraction shown in Expression (2) described above in the soft part image Gs, thereby generating the muscle image Gm with the muscle tissue of the subject.

In next Step S106, as described above, the specification unit 48 specifies an imaging part of the subject and specifies an affection risk or a disease level according to the imaging part with reference to the correspondence relationship information 36 according to the specified part. For example, in a case where a thigh is specified as an imaging part as described above, the specification unit 48 refers to the correspondence relationship information 36 representing a correspondence relationship representing an affection risk of diabetes and a muscle mass of the thigh, and specifies the affection risk of diabetes corresponding to the muscle mass derived in Step S104.

In next Step S108, the specification unit 48 makes the display unit 24 display a specification result specified in Step S106. The display unit 24 may be made to also display information representing the muscle mass derived in Step S104. In a case where the processing of Step S108 ends, the present information processing ends.

In this way, the information processing apparatus 10 of the embodiment generates the soft part image Gs from the first radiographic image G1 and the second radiographic image G2, and derives the muscle mass for each pixel of the derives soft part image Gs. With this, with the information processing apparatus 10 of the embodiment, it is possible to allow a physician to ascertain a local muscle mass of a thigh, a limb, or the like of the subject, instead of the muscle mass of the whole subject.

Furthermore, in the information processing apparatus 10 of the embodiment, since a risk of a disease or a disease level is specified according to the local muscle mass of the subject, it is possible to more appropriately support medical examination of a physician, and to enable effective prevention and treatment of the disease. In addition, with the information processing apparatus 10, it is possible to provide the risk of the disease or the disease level to the subject in an easy-to-understand manner.

### Second Embodiment

Hereinafter, a second embodiment will be described in detail.

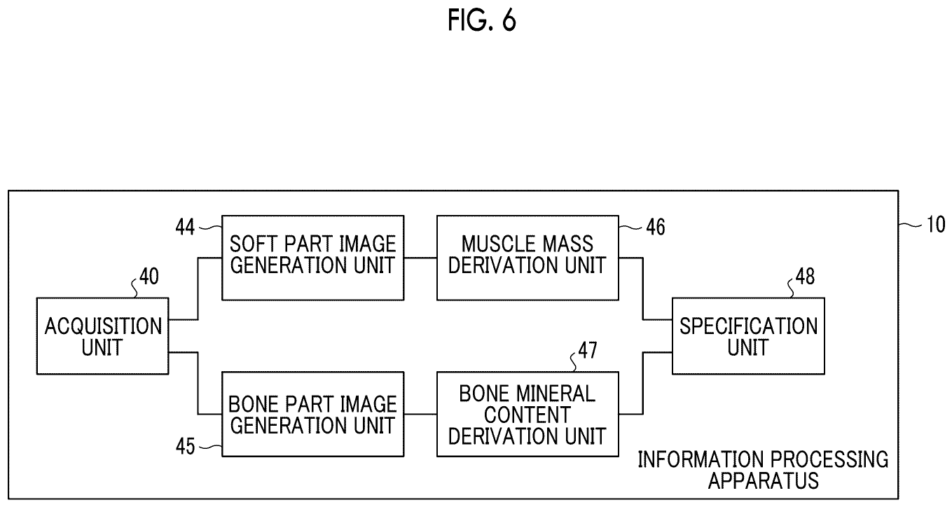

FIG. 6 is a functional block diagram showing an example of the functional configuration of an information processing apparatus 10 of the embodiment. The information processing apparatus 10 of the embodiment is different from the information processing apparatus 10 (see FIG. 3) of the first embodiment in that the information processing apparatus 10 further comprises a bone part image generation unit 45 and a bone mineral content derivation unit 47.

The bone part image generation unit 45 generates a bone part image Gb representing a bone part region with a bone tissue of the subject W from the first radiographic image G1 and the second radiographic image G2 acquired by the acquisition unit 40.

As an example, the bone part image generation unit 45 of the embodiment performs weighting subtraction between corresponding pixels as shown in Expression (4) described below for the first radiographic image G1 and the second radiographic image G2, thereby generating the bone part image Gb in which only a bone tissue of the subject W included in the first radiographic image G1 and the second radiographic image G2 is extracted. In Expression (4) described below, μb is a weighting factor, and x and y are coordinates of each pixel of the bone part image Gb.

$$GB ( x , y ) = G 1 ( x , y ) - μ b \times G 2 ( x , y ) \qquad ( 4 )$$

Since scattered ray components are included in each of the first radiographic image G1 and the second radiographic image G2 in addition to primary ray components of radiation transmitted through the subject W, as described above in the processing in which the soft part image generation unit 44 generates the soft part image Gs, it is preferable to eliminate the scattered ray components from the first radiographic image G1, the second radiographic image G2, and the bone part image Gb.

The bone mineral content derivation unit 47 derives a bone mineral content B for each pixel of the bone part image Gb. In the embodiment, the bone mineral content derivation unit 47 derives the bone mineral content B by converting each pixel value of the bone part image Gb into a pixel value of a bone image in a case of being acquired under a reference imaging condition. More specifically, the bone mineral content derivation unit 47 derives the bone mineral content B by correcting each pixel value of the bone part image Gb using a correction coefficient acquired from a look-up table (not shown) described below.

Here, as the tube voltage in the radiation source 3 is higher and radiation emitted from the radiation source 3 has higher energy, contrast of a soft tissue and a bone tissue in a radiographic image becomes smaller. Furthermore, in a process in which radiation is transmitted through the subject W, a low-energy component of radiation is absorbed by the subject W, and beam hardening that radiation increases in energy occurs. The increase in energy of radiation due to beam hardening becomes greater as the body thickness of the subject W is greater.

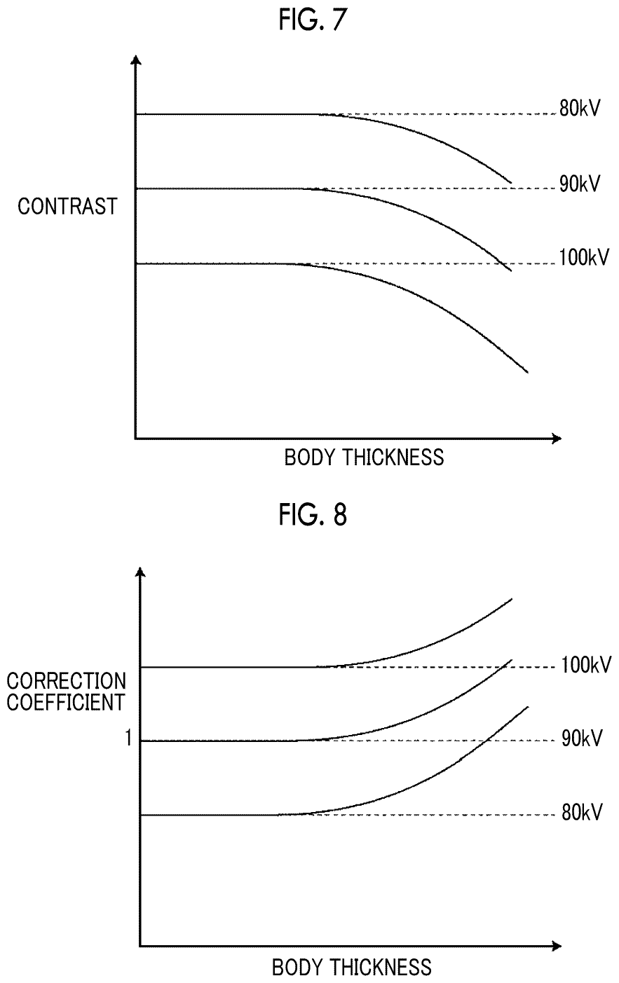

FIG. 7 is a diagram showing a relationship between the body thickness of the subject W and contrast of a bone tissue and a soft tissue. FIG. 7 shows a relationship between the body thickness of the subject W and the contrast of the bone tissue and the soft tissue at three tube voltages of 80 kV, 90 kV, and 100 kV. As shown in FIG. 7, the contrast becomes lower as the tube voltage is higher. In a case where the body thickness of the subject W exceeds a certain value, the contrast becomes lower as the body thickness is greater. As a pixel value of a bone part region in the bone part image Gb is greater, the contrast of the bone tissue and the soft tissue becomes higher. For this reason, the relationship shown in FIG. 7 is shifted to a higher contrast side as the pixel value of the bone part region in the bone part image Gb is greater.

In the embodiment, the look-up table (not shown) for acquiring the correction coefficient for correcting a difference in contrast according to the tube voltage at the time of imaging and a decrease in contrast due to the influence of beam hardening in the bone part image Gb is stored in the storage unit 22. The correction coefficient is a coefficient for correcting each pixel value of the bone part image Gb.

FIG. 8 is a diagram showing an example of the look-up table stored in the storage unit 22. In FIG. 8, a look-up table in which a reference imaging condition of a tube voltage 90 kV is set is illustrated. As shown in FIG. 8, in the look-up table, as the tube voltage is greater, and as the body thickness of the subject is greater, a greater correction coefficient is set. In the example shown in FIG. 8, since the reference imaging condition is the tube voltage 90 kV, in a case where the tube voltage is 90 kV and the body thickness is 0, the correction coefficient is 1. In FIG. 8, although the look-up table is shown in a two-dimensional manner, the correction coefficient is different according to the pixel value of the bone part region. For this reason, the look-up table actually becomes a three-dimensional table in which an axis representing the pixel value of the bone part region is added.

The bone mineral content derivation unit 47 extracts the body thickness distribution T(x,y) of the subject W and a correction coefficient C0(x,y) of each pixel according to imaging conditions including a set value of the tube voltage stored in the storage unit 22 from the look-up table. Then, as shown in Expression (5) described below, the bone mineral content derivation unit 47 derives a bone mineral content B(x,y) of each pixel of the bone part image Gb by multiplying each pixel (x,y) of the bone part region in the bone part image Gb by the correction coefficient C0(x,y). The bone mineral content B(x,y) derived in this way represents a pixel value of a bone tissue of a bone part region included in a radiographic image that is acquired by imaging the subject W at the tube voltage of 90 kV as the reference imaging condition, and from which the influence of beam hardening is eliminated.

$$B ( x , y ) = C 0 ( x , y ) \times G b ( x , y ) \qquad ( 5 )$$

To the specification unit 48 of the embodiment, information representing the muscle mass is input from the muscle mass derivation unit 46, and information representing the bone mineral content is input from the bone mineral content derivation unit 47. The specification unit 48 specifies a fall rate (fall occurrence rate) of the subject based on the muscle mass of the predetermined part derived by the muscle mass derivation unit 46, the bone mineral content derived by the bone mineral content derivation unit 47, and the correspondence relationship information 36.

It is known that a probability of a person falling and muscle or muscle strength generally have a correlative relationship. In particular, it is known that a probability of a person falling and a mass of muscle (soleus muscle) of a calf as a part of a lower limb, muscle (gluteus maximus muscle and gluteus medius muscle) supporting a pelvis, or muscle strength of the muscle have a correlative relationship.

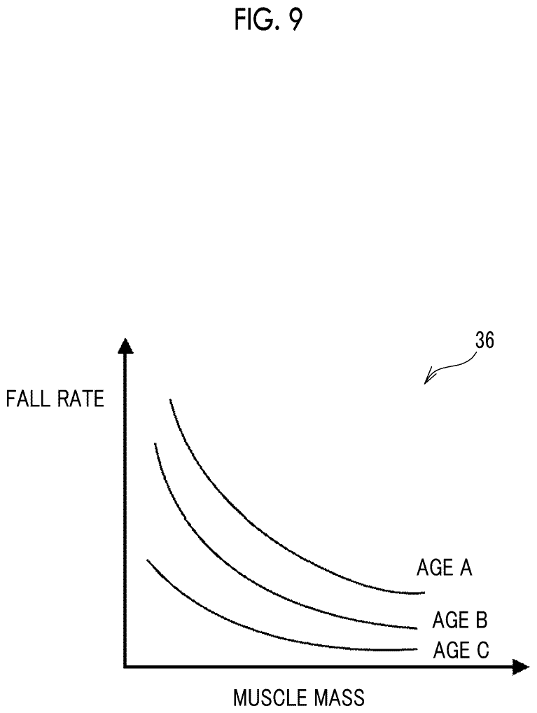

As an example, FIG. 9 shows correspondence relationship information 36 representing a correlative relationship between a muscle mass and a fall rate for each age. In the correspondence relationship information 36 shown in FIG. 9, at every age, as the muscle mass is smaller, the fall rate becomes higher. Even though the muscle mass is the same, as the bone mineral content is smaller, the fall rate becomes higher, and a probability of the person becoming fractured in a case of falling becomes higher. For this reason, the information processing apparatus 10 of the embodiment has the correspondence relationship information 36 of each age shown in FIG. 9 for each bone mineral content, and a plurality of pieces of correspondence relationship information 36 are stored in the storage unit 22.

Though also depending on muscle quality, muscle strength tends to be greater as the muscle mass is greater. For this reason, muscle strength for specifying the fall rate, instead of the muscle mass, may be used as a parameter.

In this way, the specification unit 48 of the embodiment specifies the fall rate of the subject using the correspondence relationship information 36 according to the part (imaging part) of the subject captured in the first radiographic image G1 and the second radiographic image G2.

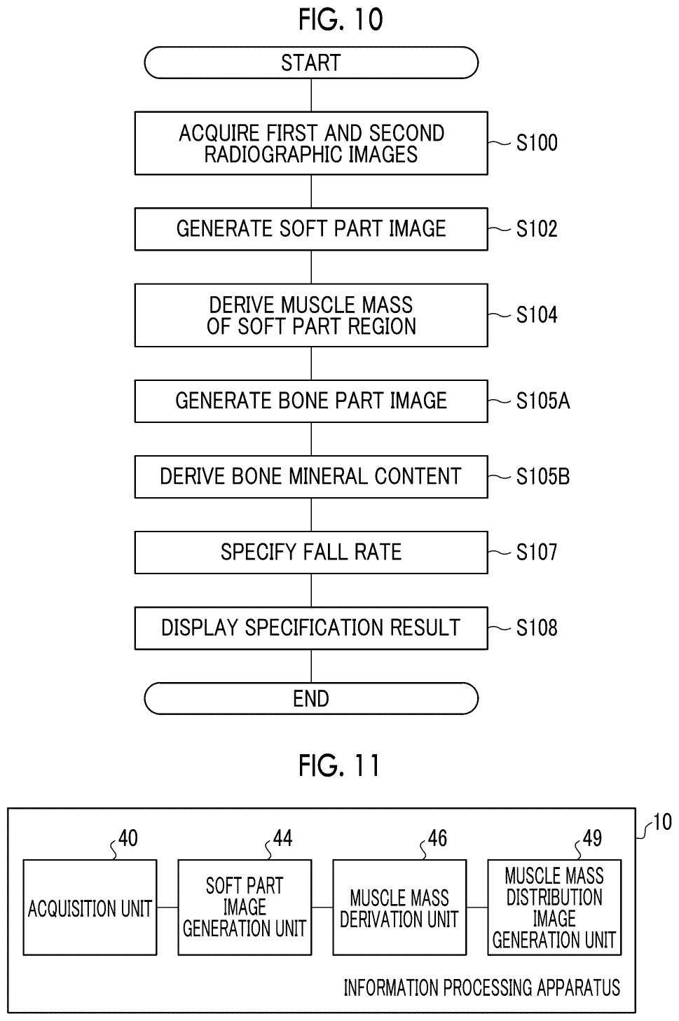

Next, the operation of the information processing apparatus 10 of the embodiment will be described. FIG. 10 is a flowchart showing an example of a flow of information processing that is executed by the CPU 20 executing the information processing program 30. The information processing of the embodiment is different from the information processing (see FIG. 5) of the first embodiment in that the information processing includes Steps S105A, S105B, and S107, instead of Step S106, between Steps S104 and S108.

In Step S105A shown in FIG. 10, as described above, the bone part image generation unit 45 performs weighting subtraction shown in Expression (4) described above between the corresponding pixels in the first radiographic image G1 and the second radiographic image G2, thereby generating the bone part image Gb with the bone tissue of the subject.

Next, in Step S105B, as described above, the bone mineral content derivation unit 47 derives the bone mineral content of the subject using the correction coefficient C0(x,y) of each pixel according to Expression (5) described above.

For this reason, in next Step S107, as described above, the specification unit 48 specifies the fall rate according to the muscle mass derived in Step S104 with reference to the correspondence relationship information 36 according to the bone mineral content derived in Step S105B.

In next Step S108, the specification unit 48 makes the display unit 24 display a specification result specified in Step S107. The display unit 24 may be made to also display the muscle mass derived in Step S104 or information representing the bone mineral content derived in Step S105B. Furthermore, in a case where the fall rate is equal to or greater than a predetermined threshold value, the display unit 24 may be made to display information representing a warning. In a case where the processing of Step S108 ends, the present information processing ends.

In this way, the information processing apparatus 10 of the embodiment generates the soft part image Gs from the first radiographic image G1 and the second radiographic image G2, and derives the muscle mass and the bone mineral content for each pixel of the soft part image Gs. With this, with the information processing apparatus 10 of the embodiment, it is possible to allow a physician to ascertain a local muscle mass of a thigh, a limb, or the like of the subject, instead of the muscle mass of the whole subject and to ascertain the bone mineral content.

Furthermore, in the information processing apparatus 10 of the embodiment, since the fall rate of the subject is specified according to the local muscle mass of the subject and the bone mineral content, it is possible to provide training effects or the like, such as restraint of the occurrence of fall or fall prevention, in an easy-to-understand manner.

### Third Embodiment

Hereinafter, a third embodiment will be described in detail.

FIG. 11 is a functional block diagram showing an example of the function configuration of an information processing apparatus 10 of the embodiment. The information processing apparatus 10 of the embodiment is different from the information processing apparatus 10 (see FIG. 3) of the first embodiment in that the information processing apparatus 10 comprises a muscle mass distribution image generation unit 49, instead of the specification unit 48.

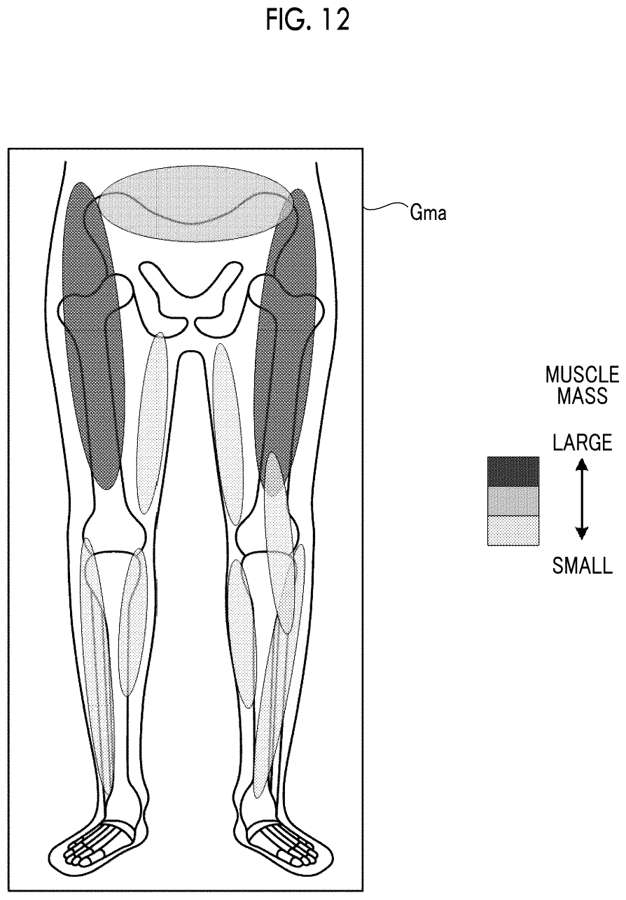

The muscle mass distribution image generation unit 49 generates a muscle mass distribution image representing a distribution of the muscle mass based on the muscle mass of each pixel of the soft part image Gs derived by the muscle mass derivation unit 46. FIG. 12 shows an example of a muscle mass distribution image Gma. The muscle mass distribution image Gma is a so-called two-dimensional map of the muscle mass. As an example, FIG. 12 shows the muscle mass distribution image Gma in which different colors are mapped to the first radiographic image G1 acquired from the first radiation detector 5 according to the muscle mass. The present disclosure is not limited to this form, and for example, mapping according to the muscle mass may be performed to the second radiographic image G2 acquired from the second radiation detector 6 or the soft part image Gs. With the muscle mass distribution image Gma, it is possible to easily ascertain an overall distribution of a muscle mass in addition to a local muscle mass in a predetermined part, for example, a part of the limb in the example of FIG. 12.

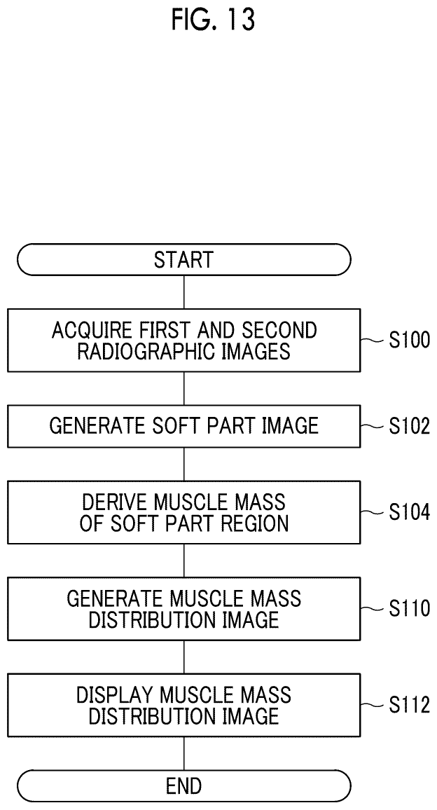

Next, the operation of the information processing apparatus 10 of the embodiment will be described. FIG. 13 is a flowchart showing an example of a flow of information processing that is executed by the CPU 20 executing the information processing program 30. The information processing of the embodiment is different from the information processing (see FIG. 5) of the first embodiment in that Steps S110 and S112, instead of Steps S106 and S108, are included.

In Step S110 shown in FIG. 13, as described above, the muscle mass distribution image generation unit 49 generates the muscle mass distribution image Gma based on the muscle mass of each pixel of the soft part image Gs derived in Step S104.

In next Step S112, the muscle mass distribution image generation unit 49 makes the display unit 24 display the muscle mass distribution image Gma generated in Step S110. The display unit 24 may be made to also display information representing the specific muscle mass derived in Step S104. In a case where the processing of Step S112 ends, the present information processing ends.

In this way, with the information processing apparatus 10 of the embodiment, since the muscle mass distribution image generation unit 49 generates the muscle mass distribution image Gma, it is possible to easily ascertain an overall distribution of a muscle mass.

As described above, the information processing apparatus 10 of each embodiment described above comprises the soft part image generation unit 44 that generates the soft part image Gs representing the soft part region with the soft tissue of the subject W from the first radiographic image G1 and the second radiographic image G2 acquired by radiation having different energy distributions transmitted through the subject W, and the muscle mass derivation unit 46 that derives the muscle mass based on the pixel value for each pixel of the soft part region of the soft part image Gs.

The information processing apparatus 10 of each embodiment described above generates the soft part image Gs from the first radiographic image G1 and the second radiographic image G2, and derives the muscle mass for each pixel of the soft part image Gs. With this, with the information processing apparatus 10 of the embodiment, it is possible to allow a physician to ascertain a local muscle mass of a thigh, a limb, or the like of the subject, instead of the muscle mass of the whole subject.

Furthermore, with the information processing apparatus 10 of each embodiment described above, since a radiographic image obtained by simple imaging, instead of a Computed Tomography (CT) image, a Magnetic Resonance Imaging (MRI) image, or the like, can be used, it is possible to ascertain a local muscle mass of the subject with a simpler apparatus.

The present disclosure is not limited to the respective embodiments described above, and for example, the respective embodiments described above may be combined. For example, a form may be made in which the specification unit 48 specifies the affection risk of the predetermined disease or the disease level of the predetermined disease based on the muscle mass of the predetermined part derived by the muscle mass derivation unit 46, the bone mineral content, and the correspondence relationship information 36. Furthermore, for example, a form may be made in which the information processing apparatus 10 comprises the specification unit 48 and the muscle mass distribution image generation unit 49.

An image that is used in deriving the muscle mass in the respective embodiments described above and in deriving the bone mineral content in the second embodiment may be a reduced image. For example, the muscle mass derivation unit 46 may derive a muscle mass for each pixel of a reduced image obtained by reducing the muscle image Gm. Furthermore, for example, the bone mineral content derivation unit 47 may derive a bone mineral content of each pixel of a reduced image obtained by the bone part image Gb. In a case where a reduced image is used in this way, since it is possible to reduce noise to improve a Signal to Noise ratio (SN ratio), it is possible to improve derivation accuracy.

In the respective embodiments described above, although the first radiographic image G1 and the second radiographic image G2 are acquired by a one-shot method, the first radiographic image G1 and the second radiographic image G2 may be acquired by a so-called two-shot method in which imaging is performed two times. In this case, the radiography system 1 may comprise one radiation detector. As the imaging conditions, any of the imaging conditions when the first radiographic image G1 is acquired and the imaging conditions when the second radiographic image G2 is acquired may be used. In a case of a two-shot method, there is a possibility that the position of the subject W included in the first radiographic image G1 and the second radiographic image G2 is deviated due to body movement of the subject W during imaging. For this reason, it is preferable that the processing of each embodiment described above is executed after registration of the subject W is performed in the first radiographic image G1 and the second radiographic image G2. As processing of registration, for example, a method described in JP2011-255060A can be used. The method described in JP2011-255060A generates a plurality of first range images and a plurality of second range images representing structures having different frequency ranges for the first radiographic image G1 and the second radiographic image G2, respectively, acquires a position deviation amount between corresponding positions in the first range image and the second range image with the corresponding frequency range, and registers the first radiographic image G1 and the second radiographic image G2 based on the position deviation amount.

As the hardware structures of processing units that execute various kinds of processing, such as the functional units of the information processing apparatus 10 in the above-described embodiments, various processors described below can be used. Various processors include a programmable logic device (PLD) that is a processor capable of changing a circuit configuration after manufacture, such as a field programmable gate array (FPGA), a dedicated electric circuit that is a processor having a circuit configuration dedicatedly designed for executing specific processing, such as an application specific integrated circuit (ASIC), and the like, in addition to a CPU that is a general-purpose processor executing software (program) to function as various processing units, as described above.

One processing unit may be configured of one of various processors described above or may be configured of a combination of two or more processors (for example, a combination of a plurality of FPGAs or a combination of a CPU and an FPGA) of the same type or different types. A plurality of processing units may be configured of one processor.

As an example where a plurality of processing units are configured of one processor, first, as represented by a computer, such as a client or a server, there is a form in which one processor is configured of a combination of one or more CPUs and software, and the processor functions as a plurality of processing units. Secondly, as represented by system on chip (SoC) or the like, there is a form in which a processor that realizes all functions of a system including a plurality of processing units into one integrated circuit (IC) chip is used. In this way, various processing units may be configured using one or more processors among various processors described above as a hardware structure.

In addition, the hardware structure of various processors is, more specifically, an electric circuit (circuitry), in which circuit elements, such as semiconductor elements, are combined.

In the above-described embodiments, although a form in which the information processing program 30 is stored (installed) in advance in the storage unit 22 has been described, the present disclosure is not limited thereto. The information processing program 30 may be provided in a form of being recorded in a recording medium, such as a compact disc read only memory (CD-ROM), a digital versatile disc read only memory (DVD-ROM), and a universal serial bus (USB). Alternatively, a form may be made in which the information processing program 30 is downloaded from an external apparatus through a network.

From the above description, it is possible to ascertain the technique related to the following supplementary item.

### Supplementary Item

An information processing apparatus comprising:
a processor; and
a memory incorporated in or connected to the processor,
in which the processor is configured to
generate a soft part image representing a soft part region with a soft tissue of a subject from a first radiographic image and a second radiographic image acquired by radiation having different energy distributions transmitted through the subject, and
derive a muscle mass based on a pixel value for each pixel of the soft part region of the soft part image.
   bone tissue

What is claimed is:

1. An information processing apparatus comprising:
a processor configured to:
generate a soft part image representing a soft part region with a soft tissue of a subject from a first radiographic image and a second radiographic image acquired by radiation having different energy distributions transmitted through the subject;
generate a muscle image from the soft part image;
obtain a reduced image from the muscle image; and
derive a muscle mass for each pixel of the reduced image corresponding to the soft part region based on a pixel value of the reduced image.

2. The information processing apparatus according to claim 1,
wherein the processor is configured to derive the muscle mass with a tissue other than a fat tissue in the soft tissue as a muscle tissue.

3. The information processing apparatus according to claim 1,
wherein the processor is configured to derive the muscle mass of a predetermined part of the subject, and
the processor is further configured to, based on correspondence relationship information representing a correspondence relationship between disease information representing an affection risk of a predetermined disease or a disease level of the predetermined disease and a muscle mass of the predetermined part and the derived muscle mass, specify the affection risk or the disease level.

4. The information processing apparatus according to claim 3,
wherein the predetermined part is a part of a lower limb,
the predetermined disease is diabetes, and
the disease information is the affection risk of the diabetes.

5. The information processing apparatus according to claim 3,
wherein the predetermined part is a part of a limb or a part of a whole body,
the predetermined disease is sarcopenia, and
the disease information is the disease level of the sarcopenia.

6. The information processing apparatus according to claim 1,
wherein the processor is configured to derive the muscle mass of a predetermined part of the subject, and
the processor is further configured to, based on correspondence relationship information representing a correspondence relationship between the muscle mass of the predetermined part or muscle information regarding muscle strength according to the muscle mass of the predetermined part and a fall rate of the subject and the derived muscle mass, specify the fall rate.

7. The information processing apparatus according to claim 6,
wherein the predetermined part is at least one of a lower limb or a pelvis.

8. The information processing apparatus according to claim 1,
wherein a first radiation detector and a second radiation detector are superimposed in an irradiation direction of radiation transmitted through the subject,
the first radiographic image is acquired by the first radiation detector, and
the second radiographic image is acquired by the second radiation detector.

9. The information processing apparatus according to claim 1,
wherein the first radiographic image is acquired by a radiation detector that is irradiated with radiation having a first energy distribution transmitted through the subject, and
the second radiographic image is acquired by the radiation detector that is irradiated with radiation having a second energy distribution different from the first energy distribution transmitted through the subject.

10. An information processing method comprising:
generating a soft part image representing a soft part region with a soft tissue of a subject from a first radiographic image and a second radiographic image acquired by radiation having different energy distributions transmitted through the subject;
generating a muscle image from the soft part image;
obtaining a reduced image from the muscle image; and
deriving a muscle mass for each pixel of the reduced image corresponding to the soft part region based on a pixel value of the reduced image.

11. A non-transitory computer-readable storage medium storing therein an information processing program that causes a computer to execute:
generating a soft part image representing a soft part region with a soft tissue of a subject from a first radiographic image and a second radiographic image acquired by radiation having different energy distributions transmitted through the subject;
generating a muscle image from the soft part image;
obtaining a reduced image from the muscle image; and
deriving a muscle mass for each pixel of the reduced image corresponding to the soft part region based on a pixel value of the reduced image.
